(19) United States
(12) Patent Application Publication
(10) Pub. No.: US 2019/0142351 A1
(43) Pub. Date: May 16, 2019

(54) CT-SCANNER WITH LARGE DETECTOR PIXELS AND/OR HYGIENIC DESIGN FOR CONTINUOUS SCANNING

(71) Applicant: Teknologisk Institut, Taastrup (DK)

(72) Inventors: Lars Bager Christensen, Gadstrup (DK); Mikkel Engbo Jorgensen, Roskilde (DK); Claus Sondergaard Jensen, Roskilde (DK)

(73) Assignee: Teknologisk Institut, Taastrup (DK)

(21) Appl. No.: 16/098,621

(22) PCT Filed: May 3, 2017
(86) PCT No.: PCT/EP2017/060480

§ 371 Date: Nov. 2, 2018

(30) Foreign Application Priority Data
May 3, 2016 (DK) PA 2016 00268

(51) Int. Cl. A61B 6/03; A22B 5/00; A61B 6/04; A61B 6/10; A61B 6/00

(52) U.S. Cl. CPC A61B 6/035; A22B 5/007; A61B 6/508; A61B 6/107; A61B 6/4423; A61B 6/0471

(57) ABSTRACT

CT-scanners for industrial use e.g. at abattoirs for scanning animal carcasses or cuttings hereof should be able to perform continuous scanning for an extended period of time and should be hygienic designed and be easy to clean. The CT scanner has an elongated tube forming a through-going opening for continuous conveying objects to be scanned through the CT-scanner and may have elongated detector pixel geometry leading to a fixed anisotropic spatial resolution. The CT-scanner is especially for scanning meat pieces to determine the quality such as the distribution of muscles and fat within the meat pieces.

The present invention relates to apparatus and methods for computed tomography (CT) scanning. More specifically, the present invention relates to industrial CT scanners and on-line methods for scanning relatively large objects or bodies, and to radiation detectors and detection methods, particularly for detecting X-rays. In particular, the invention relates to high capacity CT scanners having elongated detector pixel geometry leading to a fixed anisotropic spatial resolution. Especially the CT scanner is for scanning meat pieces to determine the quality such as the distribution of muscles and fat within the meat pieces.

The invention further relates to a computer tomographic (CT) X-ray scanning system, and more particularly to a CT scanning system having a hygienic designed, easy to clean, X-ray shielded, cabinet. The system of the invention may advantageously be used in the food production industry, especially the meat processing plants for cows, cattle, pig, sheep, turkey and other domestic, farmed animals.

### BACKGROUND OF INVENTION

Computerized tomography (CT) is a diagnostic technique using an X-ray tube, an X-ray detector and a computer/processor, where it is possible to create volumes of multiple, transverse sections of an objects body. In CT-scanning many transversal two-dimensional images are reconstructed by computer analysis of transmitted X-ray intensity.

In CT X-ray scanning of an object, X-rays are used to penetrate the object to form detector signals proportional to the projection of the linear attenuation profile of the object from different angles, measured perpendicular to the object translation. Generally, the detector comprises closely spaced detector elements facing the emission spot of the X-ray tube. The emanating beam illuminates the complete detector elements. From the projections transversal images of the internal structure of the object can be reconstructed.

The X-ray source and detector elements are mounted in a gantry so that an object being imaged with the CT scanner can be translated through a field of view (FOV) of the scanner that is located within the gantry between the scanner's X-ray source and detector. When the object is passing through the FOV, the X-ray source is operable to provide X-rays that pass through the object and are incident on the detectors. The object is usually supported on a conveyor that is movable axially along an axis, conventionally the “z-axis” of a Cartesian coordinate system, relative to the gantry to position and move the object axially through the FOV. The X-ray source and the detector are rotatable around the z-axis. The size and location of the FOV is defined by a largest circle in a plane perpendicular to the z-axis that has its center on the z-axis and for which trajectories of X-rays from the X-ray source that are detectable by the detector are substantially tangent to the circle. Conventionally, a circularly cylindrical region within the X-ray beam having its axis coincident with the z-axis and a cross section coincident with the largest circle is referred to as a CT scanner's FOV.

In many multi-slice CT scanners the detector elements in the detector are generally configured in rows and columns positioned on a circularly cylindrical surface having an axis that is parallel to the z-axis and passes through the X-ray source focal spot. Conventionally, the columns are parallel to the z-axis and the rows lie along arcs of circles that are perpendicular to the z-axis. Features of the cone beam and detector are conveniently located with respect to a sagittal plane and a transverse plane. The sagittal plane is a plane that contains the z-axis and passes through the X-ray source focal spot. The transverse plane is a plane that passes through the X-ray source focal spot and is perpendicular to the z-axis.

To image spatial features of an object, the conveyor supporting the object moves the object relative to the gantry along the z-axis to translate the object through the scanner's FOV. As the object moves through the FOV the X-ray source and detector array are rotated around the z-axis to generate projections of the object that are substantially perpendicular to the z-axis with X-rays from a plurality of different view angles. At each view angle and different axial positions along the z-axis of the imaging region, detectors in the array of detectors measure intensity of X-rays from the X-ray source that pass through the object. The intensity of X-rays measured by a given detector in the array of detectors is a function of the linear attenuation by the object composition in a solid angle of the imaging region along a helical path length, hereinafter “attenuation path”, from the X-ray source, through the imaging region slice to the given detector. The measurement provides information on composition and density of tissue in the imaging region slice along the attenuation path.

In some CT scanners an axial scan procedure is performed in which the object is moved stepwise along the z-axis to “step” the imaging region through the FOV. Following each step, the X-ray source is rotated through 360 degrees or about 180 degrees to acquire attenuation measurements for slices in the imaging region. In some CT scanners a “spiral scan” is performed in which the object is steadily translated through the gantry while the X-ray source simultaneously rotates around the object and attenuation measurements for slices in the region are acquired “on the fly”.

The attenuation measurements for slices of an imaging region of an object provided by the detectors in an axial or spiral scan are generally processed using CT reconstruction algorithms known in the art as filtered back projection algorithms to map the spatial distribution of the absorption coefficient of the imaging region as a function of position. The spatial distribution is used to display and identify internal features of the object.

CT image reconstruction algorithms are used to process attenuation data assuming that for each slice in the imaging region and for each voxel of the slice, attenuation data is acquired for solid angle for an attenuation path that passes through the voxel. To satisfy this assumption the FOV of a CT scanner used to image an objects imaging region is generally configured sufficiently large to encompass the full width of the object. If portions of an object cannot fit inside the FOV for all view angles, attenuation data is generally incomplete and artifacts may be generated in images reconstructed from the data. As a result, detector arrays of conventional CT scanners are relatively large and comprise relatively large numbers of detectors.

Typically, a row of detectors in a multi-slice CT scanner detector array has between 700-1,000 detectors and there may be as many as 64 or more rows of detectors in the array so that a detector array in a typical CT scanner may have as many as 64,000 X-ray detectors. Future CT scanners are expected to have even larger numbers of detectors. The large number of detectors requires an extensive electronic support infrastructure for signal processing and data transfer. The detectors also require complicated mechanical support systems that are configured to high tolerances that provide in addition to mechanical support, various other functions such as radiation collimation and shielding for electronics associated with the detectors. As a result, CT scanning systems are relatively complicated and expensive.

The CT-scanning system described herein below is based on and includes many of the features and processing methods as described above, but is also further developed to meet the requirements for industrial application, such as at food processing companies.

In the conventional helical-scanning type of a CT scanner, a patient couch supports the object to be scanned during the continuously translation at a constant speed through an X-ray CT gantry enclosure. In such a scanner a patient couch transports an object to be scanned in one direction first and in a second direction afterwards to fulfil the scanning and directing the object such as a human away from the scanner. Such a process is not suitable for an industrial process.

Conventionally, CT scanners are located in the X-ray department of hospitals in expensive shielded rooms, a self-shielded CT scanner has been proposed in U.S. Pat. No. 4,977,585 (Imatron Inc.). Here, the scanner has a one-opening patient tunnel in which the walls and the ends of the patient tunnel include shielding material to form a shielded enclosure in which the patient body portion is scanned. However, the shielded patient tunnel is closed in one end, leading to a problem of achieving high capacity CT scanning systems, which is required for industrial application.

WO2006/034871 (Horst Eger) describes a method for determining physiological parameters of a slaughtered animal body or piece thereof with respect to determining its commercial value and/or its processing. The method is based on an imaging method by acquiring data such as by computer tomography or nuclear spin tomography. The document does not describe the construction of CT-scanners for industrial high-speed application.

WO2006/128456 (Danish Technological Institute) describes a method for automatically determining quality characteristics of a carcass on a slaughter line. The document does not describe the construction of CT-scanners for industrial high-speed application.

WO2006/129282 (Arineta Ltd) describes a CT scanner wherein the detector array has at least one high resolution region in which detectors have a high packing density and at least one low resolution region in which detectors have a low packing density and are separated by X-ray insensitive regions substantially larger than insensitive regions resulting from septa between detectors that function to reduce detector cross talk. Such a detector panel is not suitable for high-speed industrial application.

WO2009/0146851 (Gorm Nielsen) describes a CT scanning system having an X-ray cabinet comprising X-ray shielding material. The X-ray cabinet is fully surrounding the gantry and the patient table. This scanning system is not suitable for high-speed industrial application and does not fulfil hygienic requirements in food processing industry.

WO2013/126649 (L-3 Communications Security and Detection Systems, Inc) describes a system and method for imaging objects with a sparse detector array that includes fewer detectors than conventional X-ray scanning systems. The sparse detector array is positioned to receive X-ray radiation from the at least one X-ray source after passing through an inspection area. The sparse detector array includes a plurality of rows of detector elements, wherein at least some of the plurality of rows are separated by gaps such that the at least some of the plurality of rows are non-contiguous. An iterative reconstruction process is used to determine a volumetric image of the object from the radiation measurements recorded by the detectors in the sparse detector array. The system is manufactured in a cost effective manner, but still produces accurate images, by using a sparse detector array having fewer detectors than a full detector array. The reduced number of radiation measurements resulting from the use of fewer detectors is described to be compensated, at least in part, by reconstructing volumetric images using iterative reconstruction methods. This scanning system is not suitable for high-speed industrial application and does not fulfil hygienic requirements in food processing industry.

WO2015/167585 (Empire Technology Development LLC) describes a method for assessing the quality of a piece of meat, the method comprising creating a plurality of cross-sectional images through the piece of meat e.g. by CT scanning and performing image analysis on at least one of the images to determine the arrangement of fat and lean meat within the piece of meat, where the arrangement being indicative of the quality of the piece of meat. This scanning system is not suitable for high-speed industrial application and does not fulfil hygienic requirements in food processing industry.

Typically, conventional scanners may be used for diagnostic purposes in any spatial plane of an object/patient volume and they are conventionally designed with an isotropic resolution feature/option requiring a very high rotation speed for high capacity applications.

The highly anisotropic resolution of the present invention may be achieved by arranging the detector pixels in a pattern with a high number of pixels in the transverse plane and a smaller number of pixels in the z-direction. To obtain a high capacity CT scanning system, even with a moderate rotation speed of the gantry, the individual pixels are elongated in the z-direction thus forming an anisotropic spatial resolution of the object volume with low spatial resolution in the z-direction.

The food production today faces many hygienic challenges, which cannot be solved by conventional CT-scanners. The total cost of ownership is also too high and therefore there is no or in best case a weak and risk-full business case if purchasing a human CT-scanner for application in the food industry.

Thus, there is a need for a CT scanning system specially designed to fulfil the needs of industrial food production including lower cost and hygienic design and more robust cleaning ability compared to existing human CT scanners.

### SUMMARY OF INVENTION

The present invention is a CT-scanner for industrial application and may especially be suitable for continuous examination of objects such as carcasses or meat pieces, where the overall structure of the objects is known and the variation in this structure is to be examined. The CT-scanner is constructed to minimize the construction prize and may also be produced with a hygienic design making it suitable for application within food processing companies such as at abattoirs, and may be used for on-line examination within the line e.g. at abattoirs and may especially be suitable for examination of objects where the structural variation is low in the Z-axis orientation of the objects. The capacity of the CT-scanner is preferably very high, such as scanning of 700-1000 objects each hour and delivering a processed result within few seconds.

The scanner comprises an elongated tube forming a through-going opening for continuous transporting objects to be scanned through the CT-scanner, where the elongated tube has a length and a diameter and where the length is larger than the diameter, and the elongated tube is cleaning-friendly by at least at the wall forming the through-going opening being smooth and produced without any joints or if the elongated tube is produced with joints these joints are smooth allowing easy cleaning.

The CT-scanner has a gantry with an X-ray source and a detector positioned opposite of each other relative to an object to be scanned. When in function the gantry and hereby the X-ray source and detector rotate around the object to be scanned and perform helical examination of the scanned object. When examination of meat pieces is performed the purpose may be to determine the amount and location of fat in the meat piece. In a subsequent process some of this fat may be removed.

The CT-scanner described herein is preferably for use in an industrial environment with requirements for continuous operation for an extended period of time of at least one hour, and the scanner preferably comprises a detector based on at least one array of photodiodes and which photodiodes are elongated in the direction of least resolution requirement of an object to be scanned. The elongated photodiodes are preferably located with the longitudinal directions in the translation direction for scanning objects with a coarse spatial variation in the direction of translation through the scanner.

A detector of the CT-scanner comprises a number of rows and columns such as 17 rows and 352 columns. The detector is preferably based on photodiodes comprising silicon (SI), cesium iodide (CsI) and/or gallium arsenide (GaAs), and the photodiodes may be without a scintillator material.

The CT-scanner described herein is preferably for scanning slaughtered animals or parts thereof.

The CT-scanner described herein may also be for use in an industrial environment where close proximity of operators to the scanner during operation must be allowed and where regular cleaning is required to ensure the high level of hygiene in food production, the scanner may therefore comprise an elongated tube forming a through-going opening for continuous transporting objects to be scanned through the CT-scanner, where the elongated tube has a length and a diameter and where the length is larger than the diameter, and the elongated tube is cleaning-friendly such that at least the side forming the through-going opening is smooth and produced without any joints or if the elongated tube is produced with joints these joints are smooth allowing easy cleaning.

The CT-scanner may comprise an enclosure of X-ray absorbing material substantially surrounding the elongated tube except openings of the tube forming the through-going opening.

Sealing means may be located between the elongated tube and a cabinet to avoid entrance of liquid and contaminants between the elongated tube and said cabinet.

The CT-scanner may further comprise a conveyor for transporting objects to be scanned through said scanner.

### BRIEF DESCRIPTION OF FIGURES

FIG. 1 illustrates a CT scanner according to the present invention.

FIG. 2 illustrates the detector system of the CT scanner.

FIG. 3 illustrates the elongated tube of the CT scanner with a conveyor belt extending through the tube and where the conveyor belt is in a position to be cleaned inside.

FIG. 4 illustrates a view through the elongated tube with the conveyor belt and a food piece located on the conveyor belt.

FIG. 5 illustrates a reconstructed image obtained by the CT-scanner.

### DETAILED DESCRIPTION OF THE INVENTION

An aspect of the invention relates to an X-ray CT-scanner for use in an industrial environment with requirements for continuous operation for an extended period of time of at least one hour and/or where regular cleaning is required to ensure the high level of hygiene in food production, the scanner comprises an elongated tube forming a through-going opening for continuous transporting objects to be scanned through the CT-scanner, where the elongated tube has a length and a diameter and where the length is larger than the diameter, and the elongated tube is cleaning-friendly by at least at the wall forming the through-going opening being smooth and produced without any joints or if the elongated tube is produced with joints these joints are smooth allowing easy cleaning.

In a preferred embodiment the elongated tube is produced in one piece i.e. without any joints. It may seem simple to construct a tube in one piece, however, the material should preferably be washable and if used in food companies preferably also of a material approved for food production, furthermore, as the tube may require a large diameter such as of about 80 cm in diameter, and thus a length of at least more than 80 cm, such as at least 120 cm, this requires a certain thickness of the material to make the tube stable. To make the tube hygienic the detectors are located outside the tube at the detection area of the tube, and as the tube material in the detection area preferably should have a low attenuation of X-rays this may require a thin-walled tube in the detection area. In the detection area the tube should preferably also be perfectly circular, homogeneous in thickness and have a uniform density. To produce an elongated tube fulfilling these requirements is not simple. One possibility to obtain a tube of one required thickness outside the detection area and a thinner thickness in the detection area is to produce a tube with the thickness as required outside the detector area and cut or mill away part of the outside of the tube in the detecting area to obtain the preferred thickness in the detection area. Cutting or milling away material from the outside of the tube and obtaining a detection area with the requirements as stated above is difficult.

The CT scanning system preferably includes: an object conveyor; a gantry comprising an X-ray source configured to emit an X-ray beam while rotating about an object being placed on the moving object conveyor in order to be scanned, the gantry may further comprise an X-ray detector configured to receive X-rays penetrating through the object to be scanned and further configured to provide output signals representative of the received X-rays, an X-ray cabinet comprising X-ray shielding material and an elongated tube with the going-through opening offering circumferencing cover of the conveyor. It is preferred that the X-ray cabinet fully encircles the gantry although the elongated tube may extend from the cabinet.

Here the expression elongated tube preferably refers to an X-ray transparent tube, the object to be scanned is passing through the opening of the tube during a scanning operation. According to an embodiment of the present invention, the elongated tube is forming an integrated part of the X-ray cabinet and thereby contributing to a fully sealed X-ray cabinet. For food processing application the fully sealed X-ray cabinet is preferably constructed such that the CT-scanner system as a whole constitute a unit which is non-permeable to water or vapor and is with a design and of materials which is easy to clean, hereby satisfying the requirement for equipment installed and operating in food processing companies. The cabinet is preferably watertight in each direction making it possible to hose the outside clean without water or detergent enters the cabinet.

According to an embodiment of the invention, the elongated tube may support the conveyor thus allowing the introduction of an object into the X-ray cabinet on the conveyor. Preferably, the CT scanning system of the invention may further comprise shielding means to provide an X-ray shielding closure of the elongated tube openings.

It is preferred that the elongated tube is substantially tubular or cylindrical, but it may also assume other forms, such as an elliptical, square or rectangular end form.

The elongated tube may be constructed in one piece i.e. without any joint. However one or more joints may be constructed in the area close to where the X-rays pass the tube on their way from the X-ray source and towards the detector, such that the tube in the detection area is X-ray penetrable but not X-ray penetrable in areas not in the detection area. The elongated tube is preferably having a hygienic design and constructed with material which is easy to clean and if used for scanning food is made of a material approved for food production such as polyethylene e.g. in both the detection area and outside the detection area.

According to an embodiment of the invention, the elongated tube may have a transversal dimension of at least 200 mm, e.g. at least 250 mm, such as at least 300 mm, e.g. at least 400 mm, such as at least 500 mm, such as at least 600 mm, e.g. at least 700 mm, such as at least 800 mm, e.g. at least 900 mm, such as at least 1000 mm. A preferred transversal dimension of the elongated tube for abattoir purpose at pig abattoirs is 600 to 900 mm, such as around 750 mm e.g. around 800 mm for scanning e.g. middle pieces (bacon pieces). If scanning two objects at a time the transversal dimension may be doubled. It is also within one or more embodiments of the invention that the length of the elongated tube is more than 1.05 times the transversal dimension, such as 1.5 times the transversal dimension, such as around 2 times the transversal dimension.

Preferably the elongated tube is a fixed i.e. non-moving tube making an entrance and exit to the gantry and may be longer than required for objects to pass through the gantry.

The tube may have a thickness in the area outside of the detector area of at least 15 mm, such as at least 18 mm, e.g. at least 20 mm, such as at least 22 mm, e.g. at least 25 mm. Preferably the tube has a thickness of 22 mm in the areas outside of the detector area. In the detector area the tube may be thinner than outside the detector area. The thickness in the detector area may be e.g. at least 5 mm thinner than in the areas outside of the detector area, such as at least 10 mm thinner, e.g. at least 15 mm thinner. In an embodiment the tube has a thickness of 22 mm in the area outside the detector area and 5 mm in the detector area. The thinner area in the detector area improves the penetration of the X-rays and reduces the scattering of X-rays from the tube material.

The elongated tube may in the area outside the detection area including any tube extending out of the gantry comprise an X-ray non-penetrable material such as lead (Pb) and/or tungsten (W) and/or bismuth (Bi) as part of the material constituting the tube. Also a layer of X-ray non-penetrable material may be present next to the tube wall at the side facing away from the objects to be scanned. X-ray non-penetrable material is used to capture X-rays being scattered within the system and not reaching the detector. X-ray non-penetrable material incorporated in the material constituting the tube may thus secure no X-rays escape the CT-scanner though the tube material. X-ray traps inside the material constituting the tube e.g. in the form of sections comprising X-ray non-penetrable material which sections e.g. alternating are located close to each side of the tube material may be located anywhere in the tube outside of the detection area. Preferably at least one X-ray trap is located on each side of the detection area in the material constituting the tube, such as at least two X-ray traps, e.g. at least three X-ray traps on each side of the detection area. The X-ray traps which are incorporated in the material constituting the tube may be located at staggered intervals along the length of the tube and also along the material thickness of the tube. If the tube has a thickness of e.g. 22 mm outside of the detection area, three X-ray traps on each side of the detection area may be included in the material e.g. 10, 20 and 30 cm from the detector area in the lengthwise direction of the tube and be located e.g. at a depth of 0-9 mm, 7-16 mm and 14-22 mm, respectively. Any combination of number of X-ray traps and their position in the lengthwise as well as depth in the material constituting the tube is possible. Preferably the entire area with regard to the material making up the tube is covered by an X-ray trap.

The elongated tube may be made from two parallel tubes which are closed at the ends. The specifications listed herein are if not otherwise described in respect of the tube making the corridor or transport opening for objects to be scanned, this can be a first tube. The other tube, a second tube, has a larger diameter and may be constructed of metal or a polymer. The two tubes are closed at the ends e.g. with a panel made of e.g. steel such as stainless steel which may be covered with X-ray non-penetrable material at the backside i.e. at the side turning towards the inside between the first and second tube. The X-ray non-penetrable material will absorb diffuse X-rays, and the outside of the panes is cleaning-friendly. The panel is preferably attached to the first and second tube in a manner making the system water-proof and with smooth connections such than no water can enter into the system e.g. during cleaning and the connections are easy to clean.

Preferably the tube is a single tube which may be corrugated on the outside part of the tube and smooth on the inside part making the through-going opening of the CT-scanner. In the troughs i.e. the smallest part of the corrugated outside part of the tube X-ray non-penetrable material may be located.

In a preferred embodiment the CT-scanner further comprises a detector based on at least two photodiode and which photodiodes are elongated in the direction of least resolution requirement of an object to be scanned.

The detector with photodiodes may be a central part of the CT-scanner as described herein. The photodiodes are preferably elongated and may be arranged with the long edge parallel or perpendicular to the transport direction of objects to be scanned. The detector may also comprise at least two photodiodes which may be arranged with at least one photodiode with the long edge parallel to the transport direction of objects to be scanned and at least one photodiode with the short edge perpendicular to the transport direction of objects to be scanned. More preferred arrangements of photodiodes are described herein below. The elongated detectors allow a fast scanning speed of the objects described herein. Preferably the detector comprises from hundreds to thousands of photodiodes as described below.

Image data obtained by the CT-scanner may be characterized by a fixed anisotropic resolution due to the elongated photodiodes. Such a CT-scanner may be used for examination of objects of which the structures made up of different materials in general are known, but may differ between objects, and where the overall distribution and location of the different materials of the objects should be known. Especially the CT-scanner is suitable to perform high-speed scan of objects where the variation in the Z-direction is small and the variation in the X-direction is larger. Such objects may be meat pieces with different materials such as meat (muscles), fat and optionally bones and/or skin, such as belly pieces, neck fillet, shoulder clod, loin, silverside or outside. In an industrial CT-scanning process of these meat pieces one knows in overall what to look for and therefore images of lower quality than required for medicine purpose can be accepted. The obtained scan can be used to determine the quality of the scanned objects determined e.g. by the amount and/or location of fat, meat (=muscles) and bones.

The CT-scanner as described herein may be used in different industrial environments such as in industry with food production e.g. at abattoirs, or other manufacturing companies handling biological materials such as meat pieces and where non-destructive imaging technology can be suitable to examine objects.

In food production companies speed and quality are in focus. The CT-scanner described herein may deliver image data of scanned objects at a speed similar to the line speed e.g. image data each 1-60 seconds when examining different objects passing the location of the detector in a continuous line. The image data obtained for each examined object may be used to improve the quality of the product when compared to handling the object without this examination. The quality improvement may be obtained in a subsequent process e.g. by removing fat and leaving as much at as required by a customer. Such a subsequent process may be a manual process or an automatic process where abattoir worker(s) or automatic equipment, respectively, use the result of processed image data to remove an individually determined amount of e.g. fat.

The CT-scanner is preferably for continuous operation for an extended period of time of at least one hour, such as at least two hours, e.g. at least three hours, such as at least four hours, e.g. at least five hours, such as at least six hours, e.g. at least 7 hours, such as at least 8 hours, e.g. at least 9 hours, such as at least 10 hours. Preferably the CT-scanner can operate continuous 16 hours a day in at least 5 days a week. The technology of the CT-scanner is preferably a slip-ring technology allowing the transmittal of electrical energy across a moving interface by sliding contactors of parallel conductive rings concentric to the gantry axis and hereby allowing the scan frame to rotate continuously with no need to stop between rotations to rewind system cables. The CT-scanner preferably works in a helical or spiral manner where the tube and detector continuously perform a circular movement while a conveyor belt with objects to be scanned transports these objects through the CT-scanner. Depending on the line speed the obtained data information may be processed and results may be delivered with a time interval corresponding to the time interval between CT-scanning of the objects being transported pass the detector. Such a time interval may be less than 5 min, such as less than 4 min, e.g. less than 3 min, such as less than 2 min, e.g. less than 100 sec, such as less than 80 sec, e.g. less than 60 sec, such as less than 50 sec, e.g. less than 40 sec, such as less than 30 sec, e.g. less than 20 sec, such as less than 10 sec, e.g. less than 5 sec. The CT-scanner may thus be used as an in-line measuring equipment where processed data can be used online in one or more following processing steps such as removing fat or cutting the meat piece into smaller meat pieces.

The CT-scanner as described herein is preferably constructed with an elongated tube making a continuous through-going opening such that objects to be scanned can be transported through the CT-scanner in a one-way movement and such that objects are not transported back-wards within the CT-scanner after the scanning process in respect of an object has been completed.

In an embodiment the detector of the CT-scanner is constructed such that the elongated photodiodes are located with the longitudinal directions in the translation direction for scanning objects with a coarse spatial variation in the direction of translation through the scanner. Thus the elongated photodiodes of the detector can be arranged with the longitudinal directions parallel to the direction of movement of an object to be scanned and when a conveyor belt is used to transport objects through the CT-scanner the elongated photodiodes are preferably arranged with the longitudinal direction parallel to the movement direction of the conveyor belt. The large photodiodes result in lesser photodiodes (pixels) at the detector area when compared to square photodiodes of ‘normal’ size

In an embodiment the detector may be a detector array comprising a plurality of photodiodes i.e. elements arranged in a two dimensional array of column, each disposed in the direction of the Z-axis, and rows, each disposed in a plane transverse to the Z-axis. Preferably the detector elements are designed as rectangular photodiodes arranged in straight rows and columns. A detector array may comprise at least 1 row of photodiodes and at least 200 columns of photodiodes, such as at least 2 rows and at least 250 columns of photodiodes. The number or rows of photodiodes may be between e.g. 5 to 50, such as between 8 to 40, e.g. between 10 to 30, such as between 15 to 25, e.g. 20 and combined with any number of columns between 200 and 1000, such as between 225 and 900, e.g. between 250 and 800, such as between 275 and 700, e.g. between 280 and 600, such as between 300 and 500, e.g. between 310 and 400, such as between 325 and 375. In a preferred embodiment the number or rows are 17 and the number of columns are 352. The number of detector elements or pixels is preferably lower than in medical CT scanners of today.

The detector may be constructed of modules. A module may comprise e.g. 17 rows and 32 columns of the elongated photodiodes.

The dimensions of the elongated or rectangular photodiodes i.e. elements of the detector may each be between 5 and 25 mm long, such as 6-20 mm, e.g. 7-18 mm, such as 8-16 mm, e.g. 9-14 mm, such as 10-12 mm, e.g. about 11 mm, and between 2-5 mm width, such as 2.5-4.5 mm, e.g. 3-4 mm, such as about 3.5 mm. In a preferred embodiment the dimension of the rectangular photodiodes is 3.5 mm width and 11.3 mm long.

In a preferred embodiment the photodiodes in the detector are arranged in 17 rows and 352 columns and each photodiode has a dimension of 3.5 mm in width and 11.3 mm in length. The photodiodes may be arranged on boards such that the detector may comprise 11 boards each with photodiodes arranged in 17 rows and 32 columns.

Objects to be scanned by the CT-scanner described herein are preferably objects which have a low variation in the longitudinal direction which is parallel to the Z-axis during scanning and a larger variation in the transversal direction i.e. in the X- and Y-axes. The detector elements or photodiodes are made longitudinal in the Z-axis to optimize the lighting effect i.e. more radiation photons reaches each photodiode this also reduces the number of inputs required for each detector. The reduced number of inputs in the Z-direction and a higher number of inputs in the X-/Y-direction corresponds to the higher variation of the objects to be scanned in the X-/Y-direction than in the Z-direction, hereby also the velocity of the objects to be scanned can be increased. At the same time the blur of raw data is kept at a minimum.

The elongated photodiodes each absorb more X-rays than smaller photodiodes making it possible to reduce the amount of emitted X-rays, hereby using lesser X-ray current and/or high voltage current. Hereby it also becomes easier to maintain security. The elongated photodiodes also makes it possible to examine objects at a high capacity i.e. with a high gantry rotation velocity and a high conveyor velocity and still obtaining a good data or image quality for industrial purposes. The elongated photodiodes are cost effective.

The X-ray source of the CT-scanner described herein is preferably a fixed anode X-ray tubes of 90-140 keV, and 2 kW, and requiring low current intensity of 20-100 mA. The X-ray source preferably emits the X-rays in a cone beam formation of 60° in the width and 11° in the length (i.e. in the Z-axis). The X-ray source is preferably constructed with a cooling system to remove the generated heat. The cooling system is further described elsewhere herein.

The detector may comprise silicon (SI), cesium iodide (CsI) and/or gallium arsenide (GaAs). Silicon may be preferred for higher energy levels as when scanning meat, whereas cesium iodide may be more sensitive to lower energy levels. The detector of the CT-scanner as described herein preferably comprises silicon photodiodes. The silicon photodiodes have a low attenuation of the X-rays.

In a preferred embodiment the photodiodes are without a scintillator material. The registered data is raw CT-data in the form of the X-rays reaching the detector such as attenuated X-rays passing the object to be scanned or un-attenuated X-rays where the X-rays do not pass the object. The reconstruction of data is thus preferably based on conversion/translation from the obtained raw CT-data i.e. from sinograms to processed images. The detectors without scintillator are simpler in construction, give increased flexibility, are cheaper to produce and result in lesser after-glow, reduced optical cross-talk and therefore increased image quality.

Preferably the detectors are not charge-coupled device (CCD) detectors.

The CT-scanner is preferably constructed as a movable unit and where the conveyor system is also movable either as an integrated unit or as an independent unit. The CT-scanner may hereby be located at the locations where e.g. continuous scanning of objects is required. The cabinet around the CT-scanner is preferably not X-ray penetrable, securing an environment which is safety for operators. Further shielding integrated in the CT-scanner securing no escape of X-rays may be used, e.g. as described elsewhere herein. Furthermore the CT-scanner may be shielded by e.g. curtains as described elsewhere to stop diffuse radiation.

The CT-scanner is preferably constructed such that the transport direction of objects to be scanned can be either way. Any conveyor and/or conveyor belt may thus be capable of moving in a predetermined direction. Hereby a movable CT-scanner can be located anywhere in the processing line e.g. at an abattoir or another food processing factory, and the location may also be close to a wall allowing access to e.g. an operating system on only one side of the CT-scanner.

A processor may be connected to the CT-scanner and receives the registered data of X-rays during scanning of objects. The processor processes the data which when scanning carcasses or meat pieces is based on attenuation when passing fat, muscles and bones dependent on which kind of objects are being scanned. The material fat, muscles and bones have different penetrability for the X-rays. The registered raw data in the form of sinograms are reconstructed into CT images in an automatic process.

In an embodiment of the CT-scanner radiation sensitive electrical components and circuits are located outside of the area of the photodiodes where the electrical components and circuit are protected from direct radiation or are located behind protecting means shielding the electrical components and circuits from X-rays. Data such as X-ray registration in photodiodes may be transferred wireless to a remote reconstruction computer.

In an embodiment the CT-scanner further comprises a cooling system wherein a refrigerant for cooling the X-ray source may be cooled actively in a closed system, the cooling system may comprise cooling means for passing the refrigerant in the closed system through a cold environment, and where the cold environment may be cooled by passing cold liquid or air, such as dry air, into the cooling means to establish the cold environment to remove heat from the refrigerant passing the cooling means.

The X-ray source may become hot during use if not cooled thus for continuous operation for an extended period of time cooling may be necessary. A maximum temperature of about 50° C. at the X-ray source should preferably be observed.

The refrigerant for cooling the X-ray source may be in a closed system and is preferably oil.

The turning system of the gantry may also become warm during operation and a cooling system for cooling the turning system may be present. Cooling air may be obtained from an air condition system located in the production environment.

An aspect of the invention relates to use of the CT-scanner as described herein for scanning objects which has a low structural variation in the Z-axis and a higher structural variation in the X-/Y-direction when the objects are transported through the CT-scanner.

In an embodiment the object to be scanned is a slaughtered animal or a part thereof. The animal may be selected from the group of swine, cattle, cows, sheep, goats, deer, games, poultry, rabbits. The objects to be scanned may be entire carcasses, half carcasses, third parts of half carcasses, or meat pieces with different materials such as meat (muscles), fat and optionally bones and/or skin, such as belly pieces, middle pieces, neck fillet, shoulder clod, loin, silverside or outside.

Preferably the use of the CT-scanner is at an abattoir or food production factory and is for continuous scanning of objects in an in-line process. The reconstructed image and/or data can be used in a subsequent operation of cutting or trimming such as removal of fat. The examination may reveal the amount and/or location of fat and/or bones within a meat piece. Removal of fat may increase the value of a meat piece.

The examination may thus improve the possibility for an abattoir or food processing factory to produce products e.g. meat pieces with the amount of fat that is required by a single customer, such as products with a uniform amount of fat. Customers may require different amount of fat on meat pieces even of ‘similar’ meat pieces.

The objects to be scanned may vary in dimension and weight. Preferably a number of objects to be scanned are of similar kind such that processing of obtained data can use the same algorithms or the same group of algorithms when performing the calculations.

The distance between two objects in the Z-direction may be e.g. 5 cm, however, the distance may be shorter such as 4 cm, e.g. 3 cm, such as 2 cm, e.g. 1 cm. The distance may also be longer, such as 10 cm, 20 cm, 30 cm, 40 cm, 50 cm, 75 cm, 100 cm or even longer. The distance between two objects may vary e.g. due to different speed of handling the objects before the objects are loaded onto a conveyor belt transporting the objects through the CT-scanner.

Preferably the objects to be scanned are transported through the CT-scanner in a single row, however, the CT-scanner may examine objects transported in two rows through the CT-scanner.

The number of objects scanned by the CT-scanner as described herein may be at least 50 per hour, such as at least 100 per hour, e.g. at least 200 per hour, such as at least 300 per hour, e.g. at least 400 per hour, such as at least 500 per hour, e.g. at least 600 per hour, such as at least 700 per hour, e.g. at least 800 per hour, such as at least 900 per hour, e.g. at least 1000 per hour, such as at least 1100 per hour, e.g. at least 1200 per hour. The scanning may be an in-line process e.g. at an abattoir where the speed of the scanning process should be similar to the line speed which may be at least 600 animals per hours, this may require twice this number to be scanned by the CT-scanner as most animal products will be delivered from both the right and left part of the carcass. The number of objects which can be scanned may also depend on the size and/or complexity in the structure of e.g. meat pieces to be scanned.

In an embodiment the CT-scanner comprises an enclosure of X-ray absorbing material substantially surrounding the elongated tube except openings of the tube forming the through-going opening. The enclosure may constitute an X-ray cabinet comprising X-ray shielding material, shielding to a degree making the environment approved for people to be positioned close to the CT-scanner without being exposed to an increased amount of X-rays.

The present invention covers embodiments including shielding means comprising food grade shielding materials. It is also within embodiments of the invention that the elongated tube comprises layers of food grade shielding materials for obtaining the X-ray shielding. However, it is also within the scope of the invention that other suitable materials may be used for obtaining the X-ray shielding effect.

It is within an embodiment of the invention that the X-ray cabinet is arranged on a supporting structure including bearing means to support the rotation of the gantry and the supporting structure may include a number of machine mounts for levelling the X-ray cabinet.

The present invention further covers an embodiment, wherein the X-ray detector has a number of detector elements arranged to form a curved shape with the X-ray source is positioned in the center of the curvature.

In an embodiment the CT-scanner may comprise sealing means which are located between the elongated tube and a cabinet to avoid entrance of liquid and contaminants between the elongated tube and the cabinet. For the CT-scanner to be suitable to function at abattoirs and at other food processing industries it is important that the scanner can be produced in a closed and hygienic manner to comply with the safety rules and cleaning rules which should be met. The CT-scanner is preferably constructed to eliminate the risk of water in the electronic units when the CT-scanner is cleaned and to reduce the risk of growth and spreading of micro-organisms hazardous to health.

Any possible joints of the elongated tube as well as of any other external parts or parts of the CT-scanner which may come into contact with objects to be scanned, water or vapor, and/or cleaning agents, may be constructed according to acceptable hygienic levels recognized in Standards and/or Guidelines such as of the EHEDG Guidelines ‘Hygienic Equipment Design Criteria’ of e.g. April 2004; Chilled Food Association ‘Hygienic Design Guidelines’ e.g. of 2002 or International Standard ISO 14159:2002(E).

It is within one or more embodiments of the invention that one or more service panels may be provided inside the X-ray cabinet, the service panel(s) requiring tools such as doors being designed to be removed or opened for maintenance or service purposes. The doors preferably close in a water-proof manner. Doors at the side of the cabinet may have any suitable form such as squared, such doors may open outwards or may be sliding doors. Doors at each end of the cabinet i.e. where the elongated tube is visible may each include a semicircle incision corresponding to the circle-form of the tube or second tube such that it is possible to close the cabinet before working with the CT-scanner and still be able to easily enter into the cabinet for servicing. Preferably two doors are located at each end of the cabinet. The doors are preferably closed in a water-tight manner securing easy cleaning of CT-scanner system Preferably the doors are openable when a conveyor belt is located through the tube of the CT-scanner system. Easy admission to service panels even when the CT-scanner system is in position and ready to scan objects may lower the time the CT-scanner is out of function, as dismantling of the system preferably is limited.

The CT-scanner may further comprise a conveyor system such as a conveyor or conveyor belt for transporting objects to be scanned through the scanner. Such a conveyor may be a conveyor belt. The conveyor belt may be made of X-ray penetrable materials such that it does not attenuate the X-rays to a large degree. The conveyor system may have a length corresponding at least to the length of the elongated tube, preferably longer such that objects to be scanned can be loaded onto the conveyor belt and removed from the conveyor belt and/or processed before and/or after scanning outside of the area of the CT-scanner.

The width of the conveyor belt is determined according to the inner size of the tube such that the conveyor belt can be running freely i.e. without touching the inner wall of the tube.

The conveyor belt is preferably horizontal i.e. ±4°, such as ±2° in the Z-axis and preferably also in the X-axis. The horizontal level of the conveyor belt may be adjusted by a servomechanism e.g. adjustment by an automatic system where a phantom with defined angles of e.g. 60° and/or 90° such as by scanning a triangle, a cube, where the conveyor belt may get out of the horizontal level due to lifting the conveyor belt or part of the conveyor system. The adjustment of the horizontal level of the conveyor belt may also be automatic and performed during scanning of objects which are not phantoms.

The conveyor system supporting the conveyor belt may be made such that the ends of the system can be lifted to improve admittance to the entire conveyor system during cleaning. The conveyor belt may be spring loaded making it easy to be lifted.

The conveyor belt may also be removable e.g. when the conveyor is lifted in each end due to a bending function of the conveyor system or the conveyor belt may be movable without being removed from the conveyor system. Hereby it becomes easier to clean the system and reduce the risk of growth of micro-organisms.

Preferably the surface of the conveyor belt is smooth and of a material where meat and fat do not stick to the surface. The material may be polyethylene in a non-woven design such as an extruded belt. Such a surface also improves the hygiene as it is easy to clean. Preferably the conveyor belt is not influencing the X-rays i.e. is penetrable to the X-rays, whereas the holders such as the lamellas is non-penetrable to X-rays.

A processor may be connected to the CT-scanner for adjusting the speed of the conveyor belt and/or of the rotation speed of the gantry and/or regulating the X-ray source. The objects to be scanned may vary in weight and/or in the distance between the objects being transported by the conveyor belt. To avoid scanning too much empty conveyor belt, the spaces between objects to be scanned may be recognized by the system and the rotation speed of the gantry may be lowered and/or the speed of the conveyor belt may be increased and/or the X-ray source may be turned down.

In the CT-scanner the conveyor or conveyor belt may be removable for easier cleaning of the elongated tube and of the conveyor or conveyor belt; and/or the conveyor system comprises opening means for opening the conveyor for easy cleaning of e.g. the conveyor system itself.

In the CT-scanner the conveyor system may be re-installable and/or closable and the scanner is easily re-calibrated using a pre-determined phantom. The re-calibration may comprise the horizontal levelling of the conveyor belt as described elsewhere herein to obtain a horizontal conveyor belt in the area where the gantry rotates around the conveyor belt.

The conveyor system may be a conveyor belt which is capable of continuously transporting objects to be scanned through the scanner e.g. by driving an endless conveyor belt by at least one driving valve outside each end of the elongated tube of the CT-scanner. No stop or rewinding of the conveyor system is needed. The conveyor belt may at the side used for transport of objects comprise carrying means for carrying the objects forward, such carrying means may be lamellas which when objects such as meat pieces are located on the conveyor belt the lamellas are located between the meat pieces.

The conveyor of the CT-scanner may have a continuous conveyor belt forming an inner volume defined by the conveyor belt which when in use has a transporting part for transporting objects to be scanned through the scanner and a returning part which returns underneath the transporting part, and the inner volume, which may be non-moving in relation to the moving conveyor belt may comprise at least two lamellas which preferably are perpendicular or deviating from this of less than 45° according to the direction of the transporting part of the conveyor belt, however, the lamellas may be orientated differently. The lamellas are preferably made of an X-ray absorbing material or material which is non-penetrable to X-rays, hereby the lamellas function as a barrier to X-rays such that X-rays cannot escape the CT-scanner system. Preferably no lamella is present in the area where the detector area of the tube is located.

Beneath the conveyor belt at least one plate which is non-penetrable to X-rays may be positioned to reduce the risk of X-rays escaping from the CT-scanner. Preferably such plate is not positioned in the area where the detector area of the tube is located.

The at least two lamellas located within the area defined by the conveyor belt may be surrounded by a smooth material which is cleaning-friendly, hereby it is possible to utilize less cleaning-friendly material for the lamellas and still obtain a cleaning-friendly conveyor system, which may be a prerequisite for installation and use of the CT-scanner at abattoirs and other food processing industries.

The at least two lamellas surrounded by a smooth material may be a unit and may be removable from the conveyor system. Preferably the lamella unit can be removed from the conveyor system in a dismantling process which is easy and fast hereby the construction improves the cleaning process.

In an embodiment the CT-scanner comprises at least one X-ray absorbing curtain or other X-ray absorbing closing mechanism located close to or at each end of the through-going opening. An X-ray absorbing closing mechanism is preferably a system which the transported objects can easily pass by, a flexible system may be preferred as the transported objects can push away a flexible system, and the flexible system may by gravitation return to the starting point e.g. hanging in a vertical or nearly vertical position when no object is passing the X-ray absorbing closing mechanism. Preferably the X-ray absorbing closing mechanism is produced of a material which is approved for food contact. The X-ray absorbing closing mechanism may be used when the CT-scanner is used in an in-line application for on-line examination of objects such as at abattoirs where the CT-scanner when in function and handling e.g. at least 500 objects per hour need to be open in both ends of the elongated tube, but X-rays may not escape from the CT-scanner.

The at least one curtain being an X-ray absorbing closing mechanism may be at least two strips, which may be attached in the upper part of the through-going opening or above the end of the through-going opening, the strips may each have a free-moving part extending downward. The strips are preferably made of an X-ray absorbing material, and may have a weight which secures fast returning of each strip to the starting point when no object is passing the X-ray absorbing closing mechanism. The number of strips forming a single X-ray absorbing closing mechanism may be any suitable numbers making it easy for objects to pass the strips and for the strips to return to the starting point, the number may thus be 3 strips, such as 4 strips, e.g. 5 strips, such as 6 strips, e.g. 7 strips, such as 8 strips, e.g. 9 strips, such as 10 strips, e.g. 11 strips, such as 12 strips or more. The number of strips need not be similar for all curtains of the CT-scanner.

The strips may each further comprises at least one magnet or material capable to attract a magnet in the free-moving part. The magnet or material in the strip which is capable of attracting a magnet may connect to or may by attracted by magnetism from a material in the area below the strip's ends when these return to the starting point. The magnetic connection or attraction without direct connection secure easy passage of transported objects together with safe closure of the X-ray absorbing closing mechanism when no object is transported pass the position of the X-ray absorbing closing mechanism. The magnetic attraction further reduces the risk of strips adhering to each other and of fluttering strips and thus reducing the risk of X-rays escaping from the CT-scanner.

For the strips to attach to the CT-scanner system, the CT-scanner system may comprise at least one magnet or material capable to attract a magnet in an area below the at least one curtain or in the conveyor system, such that the strips of the at least one curtain due to magnetism is attracted towards the conveyor and hereby shielding the opening.

The CT-scanner may have more than one X-ray absorbing closing mechanism such as curtain. The number of X-ray absorbing closing mechanisms such as curtains may be at least two at each end of the elongated tube, such as three, e.g. four, such as five, e.g. six at each end. The increasing numbers of X-ray absorbing closing mechanism such as curtains reduces the risk of X-rays escaping from the CT-scanner system. The distance between two curtains at one end of the tube may be e.g. 10 cm, such as 15 cm, e.g. 20 cm, such as 25 cm, e.g. 30 cm, such as 35 cm, e.g. 40 cm, such as 45 cm, e.g. 50 cm, such as 55 cm. The distance between two curtains may also correspond to the length of the objects to be scanned, where the length is the dimension in the Z-direction. Hereby the risk of generating openings in the curtains is reduced and thus the risk of X-ray escaping from the CT-scanner is reduced.

The curtains used for X-ray absorbing closing mechanism are preferably washable. The curtains may be washed while mounted on the CT-scanner, however, the curtains may also be demountable. Demountable curtains make it easy to change a dirty curtain to at clean curtain, such as performing a change at certain intervals e.g. each morning or when a certain number of objects have passed the curtains.

In an embodiment of the CT-scanner the elongated tube may be penetrable to X-rays at least in a detection area which is preferably a ring-formed area where the X-ray source and the detector is located. The presence of the tube between the X-ray source-detector system and the transported objects increases the hygiene of the system as the tube is preferably easy to clean.

The tube in areas outside of the detection area may be non-penetrable to X-rays and/or may be shielded by a material which is non-penetrable to X-rays. The areas of the tube which are non-penetrable to X-rays decrease the risk of X-rays escaping the CT-scanner system.

In a preferred embodiment of the CT-scanner an X-ray source is located outside of the elongated tube directing X-rays into the tube, and the X-ray source is shielded in all directions except in the direction towards the elongated tube by a material which is substantially non-penetrable to X-rays. This material may be lead. The shielding of the X-ray source decreases the risk of X-rays escaping the CT-scanner system.

In another preferred embodiment of the CT-scanner a detector is located outside of the elongated tube and opposite of the X-ray source, and the detector is shielded in all directions except in the direction towards the elongated tube by a material which is substantially non-penetrable to X-rays. This material may be lead. This shielding of the detector also decreases the risk of X-rays escaping the CT-scanner system.

Any of the embodiments related to the CT-scanner comprising elongated photodiodes may be combined with any of the embodiments related to the CT-scanner with a hygienic design as both themes may be relevant for CT-scanners used industrially e.g. for continuous scanning such as at abattoirs or in food production companies.

In a preferred embodiment the CT-scanner comprises an elongated tube forming a through-going opening for continuous conveying objects to be scanned through the CT-scanner as described herein and at detector with elongated photodiodes as further described herein. The CT-scanner may further comprise any of the characteristics described herein.

### DETAILED DESCRIPTION OF THE FIGURES

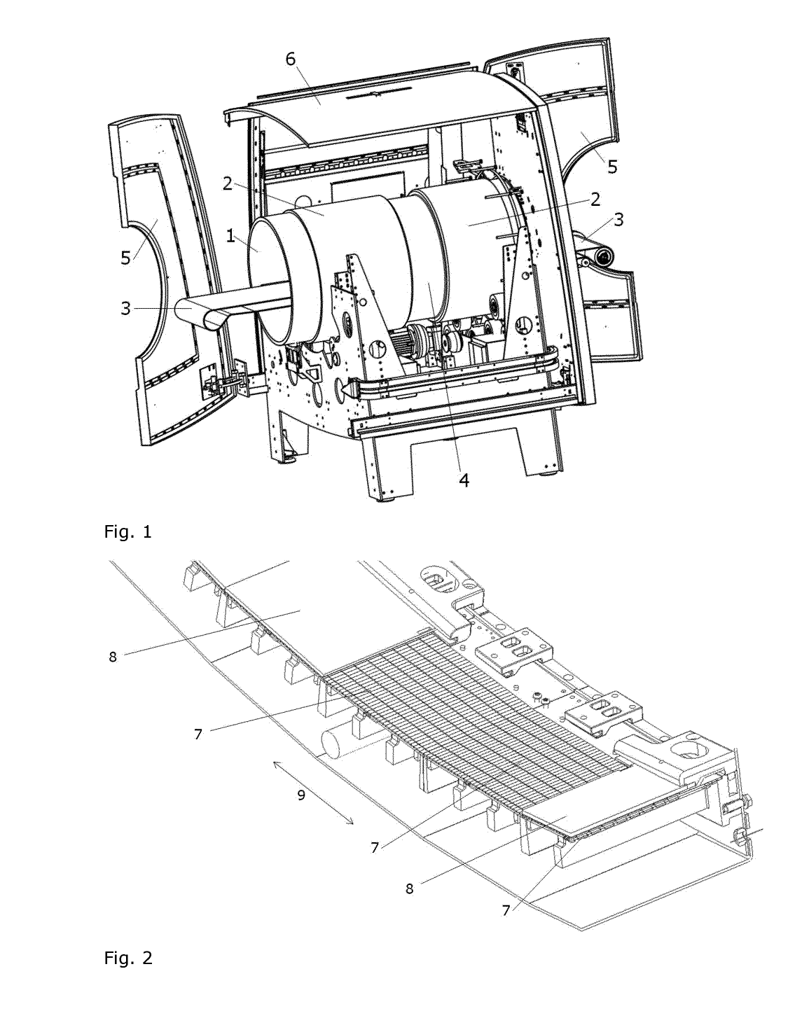

FIG. 1 illustrates a CT-scanner according to the present invention where a part of the cabinet (6) is removed. The CT-scanner has an elongated tube (1) forming a through-going opening for continuous transporting objects to be scanned through the CT-scanner. X-ray protecting material (2) surrounds the main part of the elongated tube (1) except in the area (4) where the X-ray source and detector are located and the X-ray should be able to pass through the elongated tube (1). A conveyor belt (3) is positioned in the elongated tube (1) for continuous transport of objects to be scanned through the scanner. The CT-scanner is surrounded by a cabinet (6) which when closed fully encloses the components of the CT-scanner except that the openings of the elongated tube (1) is open and end parts of the tube forming the elongated tube (1) may be present outside of the end doors (5). The sides of the CT-scanner preferably also comprise doors making it easy to get access to the components of the CT-scanner. The closed CT-scanner is preferably tight such that the CT-scanner can be washed due to requirements in food producing companies.

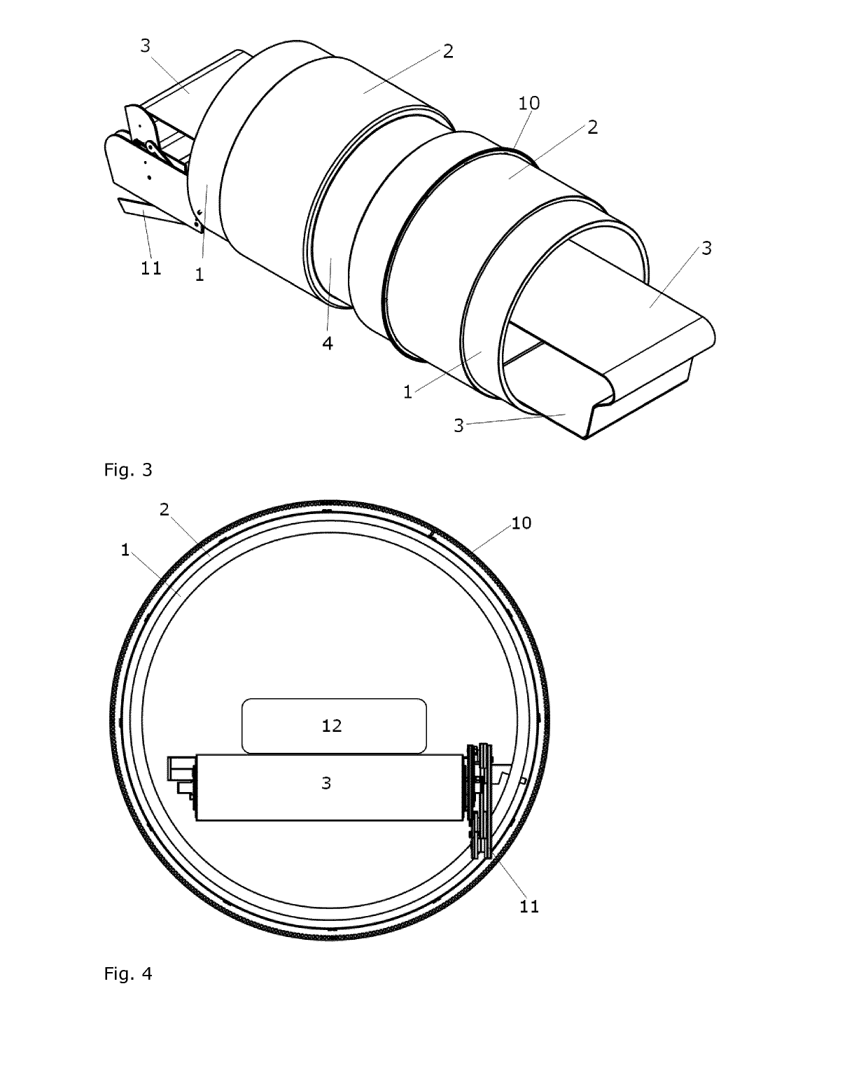

FIG. 2 illustrates a part of the detector (7) of the CT scanner. A detector comprises a number of photodiodes, which each is elongated in the direction perpendicular to the rotation direction (9). The detectors (7) may be covered with scintillator (8), here only shown covering some detectors (7).

FIG. 3 illustrates the elongated tube (1) of the CT scanner with a conveyor belt (3) extending through the elongated tube (1). The elongated tube (1) is partly surrounded by X-ray protecting material (2) except at the ends of the elongated tube (1) and at the area (4) where the X-ray source and detector are to be located outside of the elongated tube (1). Illustrated is also a conveyor belt (3) in a position to be cleaned inside. A handle (11) can be activated to shorten the total length of the conveyor belt system and extending the height of the conveyor belt system such that the conveyor belt (3) may still be running during cleaning. The system within the conveyor belt controlling the open and closed position of the conveyor belt, is not shown.

FIG. 4. illustrates a view through the elongated tube (1) of a CT scanner. Also indicated along the elongated tube (1) are X-ray protecting material (2) and the tracho ring (10). Illustrated inside the elongated tube (1) are the location of the conveyor belt (3) and a food piece (12) located on the conveyor belt (3).

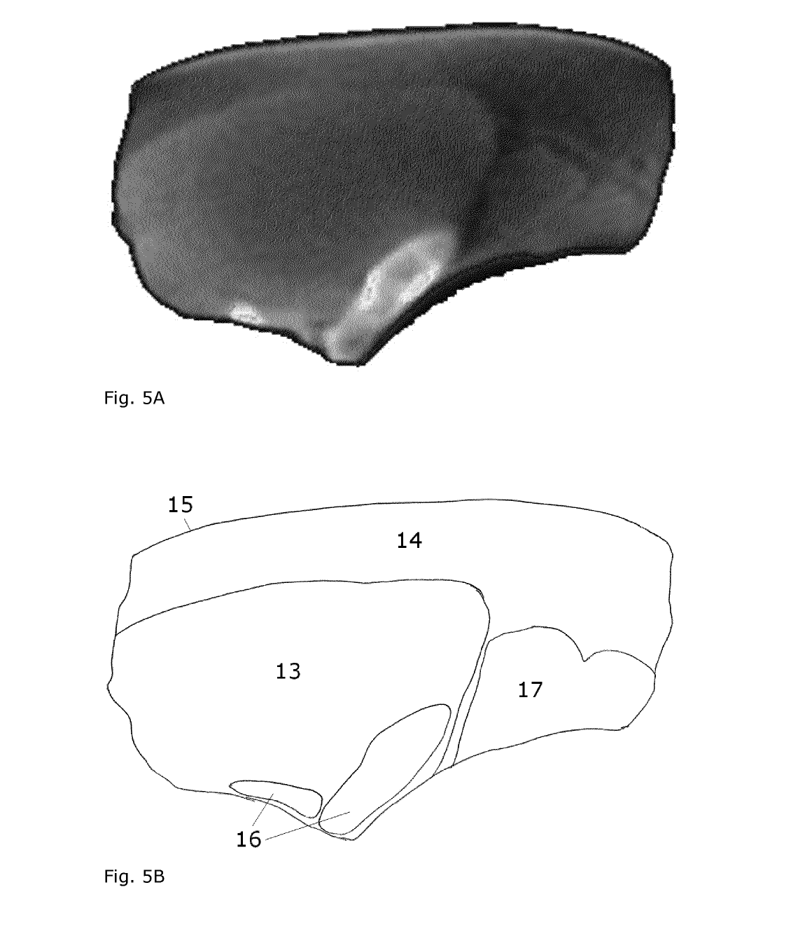

FIG. 5 illustrates an image obtained by the CT-scanner of a pork loin part. FIG. 5A is the original image in greyscales and FIG. 5B is an outline drawing of FIG. 5A indicating the different elements of the analyzed meat piece with the eye muscle (Longissimus dorsi) (13), fat (14), rind (15), bones (16) and the muscle Longissimus costarum (17).

### Items of the Invention

1. An X-ray CT-scanner for use in an industrial environment with requirements for continuous operation for an extended period of time of at least one hour, said scanner comprises a detector based on at least one array of photodiodes and which photodiodes are elongated in the direction of least resolution requirement of an object to be scanned.
2. The CT-scanner of item 1, wherein the elongated photodiodes are located with the longitudinal directions in the translation direction for scanning objects with a coarse spatial variation in the direction of translation through the scanner.
3. The CT-scanner according to any of the preceding items, wherein the elongated photodiodes are arranged with the longitudinal directions parallel to the direction of movement of an object to be scanned.
4. The CT-scanner according to any of the preceding items, wherein said detector comprises at least 1 row of photodiodes and at least 200 columns of photodiodes.
5. The CT-scanner according to any of the preceding items, wherein the object to be scanned is a slaughtered animal or a part thereof.
6. The CT-scanner according to any of the preceding items, wherein the detector is based on photodiodes comprising silicon (SI) and/or cesium iodide (CsI) and/or gallium arsenide (GaAs).
7. The CT-scanner according to any of the preceding items, wherein the photodiodes are without a scintillator material.
8. The CT-scanner according to any of the preceding items, wherein radiation sensitive electrical components and circuits are located outside of the area of the photodiodes where the electrical components and circuit are protected from direct radiation or are located behind protecting means shielding the electrical components and circuits from X-rays.
9. The CT-scanner according to any of the preceding items, further comprising a cooling system wherein a refrigerant for cooling the X-ray source is cooled actively in a closed system, said cooling system comprises cooling means for passing the refrigerant in said closed system through a cold environment, and where said cold environment is cooled by passing cold liquid or gas into said cooling means to establish the cold environment to remove heat from the refrigerant passing said cooling means.
10. The CT-scanner according to item 9, wherein said liquid or gas is selected from the group of air, dry gas, dry air, inert gas.
11. Use of the CT-scanner according to any of item 1 to 10 for scanning animal carcasses, such as half-carcasses or cuttings.

Any of the items 1-11 may be combined with any of the items 12-36 below.
12. An X-ray CT-scanner for use in an industrial environment where close proximity of operators to the scanner during operation must be allowed and where regular cleaning is required to ensure the high level of hygiene in food production, said scanner comprising
   a. An elongated tube forming a through-going opening for continuous transporting objects to be scanned through the CT-scanner, where said elongated tube has a length and a diameter and where the length is larger than the diameter, and
   b. Said elongated tube is cleaning-friendly by at least at the side forming the through-going opening being smooth and produced without any joints or if said elongated tube is produced with joints these joints are smooth allowing easy cleaning.
13. The CT-scanner according to item 12, further comprising an enclosure of X-ray absorbing material substantially surrounding the elongated tube except openings of the tube forming the through-going opening.
14. The CT-scanner according to any of the preceding items 12 to 13, wherein sealing means are located between said elongated tube and a cabinet to avoid entrance of liquid and contaminants between said elongated tube and said cabinet.
15. The CT-scanner according to any of the preceding items 12 to 14, wherein any joints of the elongated tube are made according to acceptable levels of International Standard such as ISO 14159:2002(E).
16. The CT-scanner according to any of the preceding items 12 to 15, further comprising a conveyor for transporting objects to be scanned through said scanner.
17. The CT-scanner according to any of the preceding items 12 to 16, wherein said conveyor has a length corresponding at least to the length of the elongated tube.
18. The CT-scanner according to any of the preceding items 12 to 17, wherein said conveyor is removable for easier cleaning of the elongated tube and of the conveyor and/or said conveyor comprises opening means for opening said conveyor for easy cleaning of the conveyor.
19. The CT-scanner according to any of the preceding items 12 to 18, wherein said conveyor is re-installable and/or closable and the scanner is easily re-calibrated using a pre-determined phantom.
20. The CT-scanner according to any of the preceding items 12 to 19, wherein said conveyor is capable of continuously transporting objects to be scanned through said scanner.
21. The CT-scanner according to any of the preceding items 12 to 20, wherein said conveyor has a continuous conveyor belt forming an inner volume defined by the conveyor belt which when in use has a transporting part for transporting objects to be scanned through the scanner and a returning part which returns underneath the transporting part, and said inner volume comprises at least two lamellas which are perpendicular or deviating from this of less than 45° according to the direction of the transporting part of the conveyor belt and said lamellas are made of an X-ray absorbing material.
22. The CT-scanner according to any of the preceding items 12 to 21, wherein said at least two lamellas are surrounded by a smooth material which is cleaning-friendly.
23. The CT-scanner according to any of the preceding items 12 to 22, wherein said at least two lamellas surrounded by a smooth material is a unit which is removable from the conveyor.
24. The CT-scanner according to any of the preceding items 12 to 23, further comprising at least one X-ray absorbing curtain at each end of said through-going opening and the curtains preferably is made of a material approved for food contact.
25. The CT-scanner according to any of the preceding items 12 to 24, wherein said at least one curtain comprises at least two strips, with a free-moving part extending downward.
26. The CT-scanner according to any of the preceding items 12 to 25, wherein said strips each further comprises at least one magnet or material capable to attract a magnet in the free-moving part.
27. The CT-scanner according to any of the preceding items 12 to 26, further comprising at least one magnet or material capable to attract a magnet in an area below said at least one curtain or in said conveyor, such that the strips of the at least one curtain due to magnetism is attracted towards said conveyor and hereby shielding said opening.
28. The CT-scanner according to any of the preceding items 12 to 27, wherein the number of curtains is at least two on each end of the elongated tube.
29. The CT-scanner according to any of the preceding items 12 to 28, wherein the curtains are washable.
30. The CT-scanner according to any of the preceding items 12 to 29, wherein the curtains are demountable.
31. The CT-scanner according to any of the preceding items 12 to 30, wherein the number of strips in each curtain is at least 5.
32. The CT-scanner according to any of the preceding items 12 to 31, wherein the tube at least in a detection area comprising a ring formed area with the location of the X-ray source and the detector is penetrable to X-rays.
33. The CT-scanner according to any of the preceding items 12 to 32, wherein said tube outside of said detection area is non-penetrable to X-rays and/or is shielded by a material which is non-penetrable to X-rays.
34. The CT-scanner according to any of the preceding items 12 to 33, wherein an X-ray source is located outside of said elongated tube directing X-rays into said tube, and wherein said X-ray source is shielded in all directions except in the direction towards the elongated tube by a material which is non-penetrable to X-rays substantially.
35. The CT-scanner according to any of the preceding items 12 to 34, wherein a detector is located outside of said elongated tube and opposite of said X-ray source, and wherein said detector is shielded in all directions except in the direction towards the elongated tube by a material which is non-penetrable to X-rays.
36. Use of the CT-scanner according to any of the preceding items 12 to 35 for scanning animal carcasses, such as half-carcasses or cuttings.

Any of the items 12-36 may be combined with any of the items 1 to 11.

### LIST OF REFERENCE SIGNS

1. Elongated tube
2. X-ray protecting material
3. Conveyor belt
4. Area of the elongated tube where the X-ray source and detector are located outside of the elongated tube
5. Doors
6. Cabinet
7. Detector with elongated photodiodes
8. Scintillator
9. Rotation direction
10. Tacho ring
11. Handle for opening and closing conveyor belt
12. Food product
13. Eye muscle, Longissimus dorsi
14. Fat
15. Rind
16. Bone
17. Longissimus costarum muscle

1. An X-ray computed tomography (CT)-scanner, the scanner comprising:
an elongated tube forming a through-going opening configured to continuously transport objects to be scanned through the CT-scanner, wherein the elongated tube comprises a length and a diameter, and wherein the length is larger than the diameter, and elongated tube is at least at the side forming the through-going opening, wherein the side is smooth and produced without any joints or the elongated tube is produced with joints that are smooth.

2. The CT-scanner according to claim 1, further comprising:
an enclosure of X-ray absorbing material substantially surrounding the elongated tube except openings of the elongated tube forming the through-going opening.

3. The CT-scanner according to claim 2, wherein the elongated tube and a cabinet are sealed together to avoid entrance of liquid and contaminants between the elongated tube and the cabinet.

4. The CT-scanner according claim 1, further comprising:
a conveyor configured to transport the objects to be scanned through the scanner.

5. The CT-scanner according to claim 4, wherein the conveyor is removable during cleaning of the elongated tube and the conveyor and the conveyor is configured to open during the cleaning of the conveyor.

6. The CT-scanner according tea claim 1, further comprising:
at least one X-ray absorbing curtain at each end of the through-going opening.

7. The CT-scanner according to claim 1, wherein a portion of the elongated tube is in a detection area comprising a ring formed area, and wherein a location of an X-ray source and a detector is permeable to X-rays.

8. The CT-scanner according to claim 7, wherein a portion of the elongated tube outside of the detection area is non-permeable to X-rays or is shielded by a material that is non-permeable to X-rays.

9. The CT-scanner according to claim 1, wherein an X-ray source is located outside of the elongated tube and configured to direct X-rays into the elongated tube, and wherein the X-ray source is shielded in all directions except in a direction facing the elongated tube by a material that is non-permeable to X-rays.

10. The CT-scanner according to claim 1, further comprising:
a detector comprising at least one array of photodiodes, and wherein the photodiodes are elongated in a direction of least resolution required of an object to be scanned.

11. The CT-scanner according to claim 10, wherein the elongated photodiodes are located in a longitudinal direction of translation through the scanner, wherein the photodiodes comprise a coarse spatial variation in the direction of translation through the scanner.

12. The CT-scanner according to claim 10, wherein the detector comprises at least 1 row of photodiodes and at least 200 columns of photodiodes.

13. The CT-scanner according to claim 10, wherein the detector comprises photodiodes comprising silicon (Si), cesium iodide (CsI), and gallium arsenide (GaAs).

14. The CT-scanner according to claim 10, wherein the photodiodes are free of a scintillator material.

15. A method of using a computed tomography (CT)-scanner for scanning animal carcasses, the method comprising:
transporting, by a conveyor, objects through the CT-scanner;
scanning the objects with a detector; and
cleaning the scanner through a side comprising a through-going opening.

16. The method of claim 15, wherein the animal carcasses comprise half-carcasses or cuttings.

17. The method of claim 15, wherein the transporting is continuos and the CT scanner comprises an elongated tube that forms the through-going opening.

18. The method of claim 15, further comprising:
removing the conveyor prior to the cleaning.

19. The method of claim 15, further comprising:
opening the conveyor prior to the cleaning.

20. The method of claim 15, wherein the detector comprises at least one array of photodiodes.
